(19) United States
(12) Patent Application Publication
(10) Pub. No.: US 2011/0301480 A1
(43) Pub. Date: Dec. 8, 2011

(54) SYSTEM AND METHOD FOR ASSESSING A LIKELIHOOD OF A PATIENT TO EXPERIENCE A CARDIAC ARRHYTHMIA

(75) Inventor: Raja N. Ghanem, Edina, MN (US)

(73) Assignee: Medtronic, Inc.

(21) Appl. No.: 12/793,344

(22) Filed: Jun. 3, 2010

(51) Int. Cl. A61B 5/0468; A61B 5/02

(52) U.S. Cl. 600/516; 600/508

(57) ABSTRACT

Systems and method for assessing a likelihood of a patient having a heart to experience a cardiac arrhythmia. A sensor is configured to detect a premature ventricular contraction of the heart, the premature ventricular contraction being premature relative to a rate of ventricular contractions during a period preceding the premature ventricular contraction. A processor is configured to determine a dispersion of a predetermined number of repolarizations of the heart of the patient occurring following the premature ventricular contraction of the heart if a compensatory pause is detected in conjunction with the premature ventricular contraction, then determine the likelihood of the patient experiencing the cardiac arrhythmia based, at least in part, on the dispersion of the plurality of repolarizations of the heart of the patient.

### FIELD

The present invention is related to apparatus and methods for the assessment of risk of a cardiac arrhythmia and especially to apparatus and methods for the assessment of risk of a cardiac arrhythmia by monitoring and/or measuring cardiac performance after a premature ventricular contraction.

### BACKGROUND

Cardiac pacemakers, cardioverters and defibrillators are well known in the art and provide important life-saving treatment and safeguards for many patients. Such implantable medical devices have long been utilized to treat patients prone to suffering ventricular or atrial arrhythmias such as ventricular tachycardia and ventricular fibrillation. Once implanted in the patient's body, the cardiac pacemaker, cardioverter or defibrillator monitors the patient's heart. If the heart enters fast ventricular tachycardia or ventricular fibrillation, the cardioverter/defibrillator may deliver cardioversion therapy to shock the heart out of the tachycardia or fibrillation and return the heart to normal sinus rhythm.

Determining which patients may be effectively served by the implantation of an implantable cardioverter/defibrillator may be difficult. Historically, only patients who had previously suffered ventricular fibrillation were implanted with a cardioverter/defibrillator. Subsequent clinical testing and clinical trials have provided expanded indications for patients who may benefit from a cardioverter/defibrillator. However, these indications have typically been limited to patients who had suffered a previous medical condition, such as a myocardial infarction or heart failure. As such, a substantial portion of the population which has never suffered a ventricular fibrillation episode or other traumatic cardiac event has relatively few means for being indicated for an implantable cardioverter/defibrillator.

Moreover, even to the extent that some prospective beneficiaries of an implantable cardioverter/defibrillator have suffered from a previous traumatic cardiac event, it has still often been challenging to determine if the prospective beneficiary would affirmatively benefit from receiving an implantable device. Certain characteristics of the patient, such as ejection fraction during the onset of heart failure, may provide relatively clear indications of the benefit of a cardioverter/defibrillator. However, many patients who have a traumatic cardiac event and who ultimately suffer from an arrhythmia do not, prior to the onset of the arrhythmia, have a clear indication of a likely future benefit of an implantable device.

It is known that patients, irrespective of whether they have suffered a prior cardiac episode or not, may still experience a ventricular or atrial arrhythmia such as ventricular tachycardia or ventricular fibrillation. Research has been directed toward analyzing cardiac signals to identify characteristics indicative of an increased propensity toward suffering ventricular arrhythmias such as ventricular tachycardia or ventricular fibrillation, and variously atrial arrhythmias. Such characteristics include, for instance, the electrophysiological properties of cardiac tissue or triggers that may tend to cause a ventricular tachycardia or ventricular fibrillation. However, the results of such research have proven only partially successful, as the results of the studies have tended to show that a particular cardiac characteristic will tend to show only one aspect of the underlying cause of a future ventricular or atrial arrhythmia such as ventricular tachyarrhythmia or ventricular fibrillation. Thus, the tests based on cardiac characteristics have tended to provide a substantially incomplete estimation of the patient's likelihood of suffering a ventricular or atrial arrhythmia such as ventricular tachycardia or ventricular fibrillation.

### SUMMARY

In order to fit or equip patients who could be helped by a cardiac pacemaker, cardioverter and/or defibrillator, it would be desirable to have a better accuracy of an indication of which patient or patients are most at risk of ventricular or atrial arrhythmia such as fast ventricular tachycardia and/or ventricular fibrillation.

While prior techniques exist that attempt to identify patients who may be at risk of ventricular or atrial arrhythmia such as fast ventricular tachycardia and/or ventricular fibrillation, assignment of cardiac pacemaker, cardioverter and/or defibrillator resources could be greatly enhanced if procedures for risk assessment of patients at risk of ventricular or atrial arrhythmia such as fast ventricular tachycardia and/or ventricular fibrillation could be improved. For example, if it could be established with greater likelihood that a patient was at higher risk for ventricular or atrial arrhythmia such as fast ventricular tachycardia and/or ventricular fibrillation, i.e., a patient who could be helped by a cardiac pacemaker, cardioverter and/or defibrillator, then that patient could be assigned a greater likelihood of obtaining a cardiac pacemaker, cardioverter and/or defibrillator.

Premature ventricular contractions, or “PVC's” as they are known in the art, are identified when a ventricular depolarization and contraction occurs earlier in time than would be expected given an underlying heartbeat. Owing to a compensatory pause which commonly but not always follows a premature ventricular contraction, the blood pressure of the patient may tend to fall, at least momentarily. It has been determined that when the blood pressure in a subject is at least momentarily relatively low, such as following a premature ventricular contraction with a compensatory pause, certain characteristics of a patient's cardiac performance may have an elevated indication of future propensity for cardiac arrhythmias.

In particular, it has been determined that when a premature ventricular contraction occurs followed by a compensatory pause, a patient may experience sympathetic enervation followed by vagal enervation, which may result in an immediate acceleration of the heart rate of the patient to compensate for reduced blood pressure. Sympathetic and vagal enervation may be followed by a gradual reduction in the heart rate to levels seen prior to the premature ventricular contraction. In addition, pulse alternans may tend to occur following a pause inducing premature ventricular contraction. The alternans may tend to manifest themselves as an alternating characteristic of T-waves of cardiac complexes in the heart beats which follow the premature ventricular contraction. In particular, to the extent that a measurement of T-wave alternans in cardiac beats following a premature ventricular contraction with a compensatory pause do not suggest adequately high dispersions, the patient may be indicated as being at risk of future cardiac arrhythmias, which could result in sudden cardiac death.

Further, when the premature ventricular contraction shows abnormal autonomic reflex in the patient, such as a baroreflex response, the data pertaining to the dispersions of the repolarization of the ventricles following the premature ventricular contraction may be particularly significant. Such abnormal autonomic reflex may be identified on the basis of a frequency of premature ventricular contractions in the patient over time. Alternatively, an abnormal autonomic reflex may be identified by identifying heart rate turbulence in the patient based on the detection of a premature ventricular contraction.

Devices for the collection of various kinds of cardiac data, such as Holter monitors for the collection of electrical data, are known in the art. Further, implantable sensors have been developed which allow for cardiac monitoring in a manner similar to that of a Holter monitor but without the ongoing inconvenience to the patient created by external devices. In addition, implantable cardiac therapy devices such as pacemakers, defibrillators and the like have long been provided with the capacity to sense and store cardiac data for subsequent analysis as well as to transmit diagnostic data telephonically or in real time. Any or all such devices may be utilized to sense cardiac signals and evaluate them for dispersions in repolarizations during periods of low blood pressure and abnormal autonomic reflex.

In an embodiment, a system for assessing a likelihood of a patient having a heart to experience a cardiac arrhythmia has a sensor configured to detect a premature ventricular contraction of the heart, the premature ventricular contraction being premature relative to a rate of ventricular contractions during a period preceding the premature ventricular contraction and a processor. The processor is configured to determine a dispersion of a predetermined number of repolarizations of the heart of the patient occurring following the premature ventricular contraction of the heart if a compensatory pause is detected in conjunction with the premature ventricular contraction, then determine the likelihood of the patient experiencing the cardiac arrhythmia based, at least in part, on the dispersion of the plurality of repolarizations of the heart of the patient.

In an embodiment, the likelihood of the patient experiencing the cardiac arrhythmia is relatively large when the dispersion of the predetermined number of repolarizations is relatively small.

In an embodiment, the likelihood is relatively greater based, at least in part, on a elevation of sympathetic enervation of the heart, and wherein the processor is configured to identify the elevation of sympathetic enervation of the heart combined with reduced dispersion of the predetermined number of repolarizations.

In an embodiment, the predetermined number of repolarizations occurs immediately following the premature ventricular contraction of the heart.

In an embodiment, the predetermined number of repolarizations is a predetermined number of T-waves of a cardiac complex of the heart, and wherein the determining a dispersion step is based, at least in part, on an alternating characteristic of the predetermined number of T-waves.

In an embodiment, the predetermined number of repolarizations is a predetermined number of QRST complexes of a cardiac complex of the heart, and wherein the determining a dispersion step is based, at least in part, on at least one of a total voltage, i.e., an area under an electrocardiogram curve, delivered during each of the predetermined number of QRST complexes, a variability of a total voltage delivered during a T-wave of each of the predetermined number of QRST complexes of the cardiac complex and a variability of an interval duration between a Q-wave and the T-wave of each of the predetermined number of QRST complexes of the cardiac complex.

In an embodiment, the predetermined number of premature ventricular contractions is at least five over a period of time of approximately twenty-four hours.

In an embodiment, the processor is further configured to return to detecting the premature ventricular contraction after determining the likelihood to detect further premature ventricular contractions. The processor determines the dispersion of the predetermined number of repolarizations of the heart following each of the premature ventricular contractions of the heart. The processor is further configured to determine the likelihood further based, at least in part, on the dispersion of the predetermined number of the plurality of repolarizations of the heart of the patient when the heart rate is relatively higher to the exclusion of the dispersion of the predetermined number of the plurality of repolarizations of the heart of the patient occurring when the heart rate is relatively lower.

In an embodiment, the processor is configured to return to detecting the premature ventricular contraction after determining the likelihood to detect further premature ventricular contractions. The processor determines the dispersion of the predetermined number of repolarizations of the heart following each of the premature ventricular contractions of the heart. The processor is further configured to determine the likelihood further based, at least in part, on an average of the dispersion of the predetermined number of the plurality of repolarizations of the heart of the patient when the heart rate is taken over a plurality of a predetermined number of repolarizations of the heart of the patient.

In an embodiment, the processor detects the premature ventricular contraction based, at least in part, on an interval between an R-wave of the premature ventricular contraction and an R-wave of an immediately preceding cardiac beat being not more than approximately eighty percent of an interval corresponding to the rate of ventricular contractions during a period preceding the premature ventricular contraction.

In an embodiment, the rate of ventricular contractions is based, at least in part, on an interval between R-waves of two cardiac beats immediately preceding the premature ventricular contraction.

In an embodiment, the rate of ventricular contractions is based, at least in part, on an average interval between R-waves preceding the premature ventricular contraction.

In an embodiment, the premature ventricular contraction is associated with the compensatory pause if an interval between an R-wave of the premature ventricular contraction and an R-wave of a cardiac beat immediately following the premature ventricular contraction is not less than approximately one hundred twenty percent of an interval corresponding to the rate of ventricular contractions during a period preceding the premature ventricular contraction.

In an embodiment, the rate of ventricular contractions is based, at least in part, on an interval between R-waves of two cardiac beats immediately preceding the premature ventricular contraction.

In an embodiment, the rate of ventricular contractions is based, at least in part, on an average interval between R-waves preceding the premature ventricular contraction.

In an embodiment, the predetermined number of depolarizations is within four of sixteen.

In an embodiment, the predetermined number of depolarizations is sixteen.

In an embodiment, a system for assessing a likelihood of a patient having a heart to develop a cardiac arrhythmia has a sensor configured to sense T-wave changes following a premature ventricular contraction of the heart that introduces a baroreflex response, the premature ventricular contraction being premature relative to a rate of ventricular contractions during a period preceding the premature ventricular contraction and a processor. The processor is configured to determine the likelihood of the patient experiencing the cardiac arrhythmia with the processor based, at least in part, on an alternating characteristic of a predetermined number of T-waves following the premature ventricular contraction.

In an embodiment, a system for assessing a likelihood of a patient having a heart to develop a cardiac arrhythmia comprises a sensor configured to detect a premature ventricular contraction of the heart, the premature ventricular contraction being premature relative to a rate of ventricular contractions during a period preceding the premature ventricular contraction and a processor. The processor is configured to determine an autonomic tone of the heart, determine a dispersion of a predetermined number of repolarizations of the heart of the patient occurring following the premature ventricular contraction of the heart if a compensatory pause is detected in conjunction with the premature ventricular contraction, then determine the likelihood of the patient experiencing the cardiac arrhythmia based, at least in part, on the dispersion of the plurality of repolarizations of the heart of the patient and the autonomic tone.

In an embodiment, the autonomic tone is based, at least in part, on an elevated heart rate of the patient relative to a baseline heart rate.

In an embodiment, the autonomic tone is based, at least in part, on an average heart rate of a monitoring period of approximately twenty-four hours.

In an embodiment, the elevated heart rate is measured during the predetermined number of repolarizations of the heart of the patient.

In an embodiment, the elevated heart rate is determined by an average of the heart rate during the predetermined number of repolarizations of the heart of the patient.

In an embodiment, the elevated heart rate is determined by a highest heart rate occurring during the predetermined number of repolarizations of the heart of the patient.

In an embodiment, a method is provided for assessing a likelihood of a patient to experience a cardiac arrhythmia with a system comprising a sensor and a processor. A premature ventricular contraction of the heart is detected, the premature ventricular contraction being premature relative to a rate of ventricular contractions during a period preceding the premature ventricular contraction. Then, if a compensatory pause is detected in conjunction with the premature ventricular contraction, determining a dispersion of a predetermined number of repolarizations of the heart of the patient occurring following the premature ventricular contraction of the heart. Then, the likelihood of the patient experiencing the cardiac arrhythmia is determined with the processor based, at least in part, on the dispersion of the plurality of repolarizations of the heart of the patient.

In an embodiment, a method is provided for assessing a likelihood of a patient having a heart to experience a cardiac arrhythmia with a system comprising a sensor and a processor. T-wave changes following a premature ventricular contraction of the heart that introduces a baroreflex of the heart are evaluated, the premature ventricular contraction being premature relative to a rate of ventricular contractions during a period preceding the premature ventricular contraction. Then, the likelihood of the patient experiencing the cardiac arrhythmia is determined with the processor based, at least in part, on an alternating characteristic of a predetermined number of T-waves following the premature ventricular contraction.

In an embodiment, a method is provided for assessing a likelihood of a patient to experience a cardiac arrhythmia with a system comprising a sensor and a processor. A premature ventricular contraction of the heart is detected, the premature ventricular contraction being premature relative to a rate of ventricular contractions during a period preceding the premature ventricular contraction. Then, an autonomic tone of the heart is determined. If a compensatory pause is detected in conjunction with the premature ventricular contraction, determining a dispersion of a predetermined number of repolarizations of the heart of the patient occurs following the premature ventricular contraction of the heart. Then, the likelihood of the patient experiencing the cardiac arrhythmia is determined with the processor based, at least in part, on the dispersion of the plurality of repolarizations of the heart of the patient only if the autonomic tone is present.

### FIGURES

FIG. 1 is an image of a torso of a patient;

FIG. 2 is an image of an implantable device;

FIG. 3 is a block diagram of the implantable device of FIG. 2;

FIG. 4 is exemplary of a cardiac complex of a patient;

FIG. 5 is a depiction of a premature ventricular contraction accompanied by a compensatory pulse;

FIG. 6 is a graphical depiction of a T-wave alternans analysis using a modified moving average;

FIG. 7 is a flowchart for conducing the T-wave alternans modified moving average analysis illustrated in FIG. 6;

FIG. 8 is a flowchart for analyzing heart rate turbulence in a patient; and

FIG. 9 is a flowchart for performing an assessment of a likelihood of suffering a future arrhythmia.

### DESCRIPTION

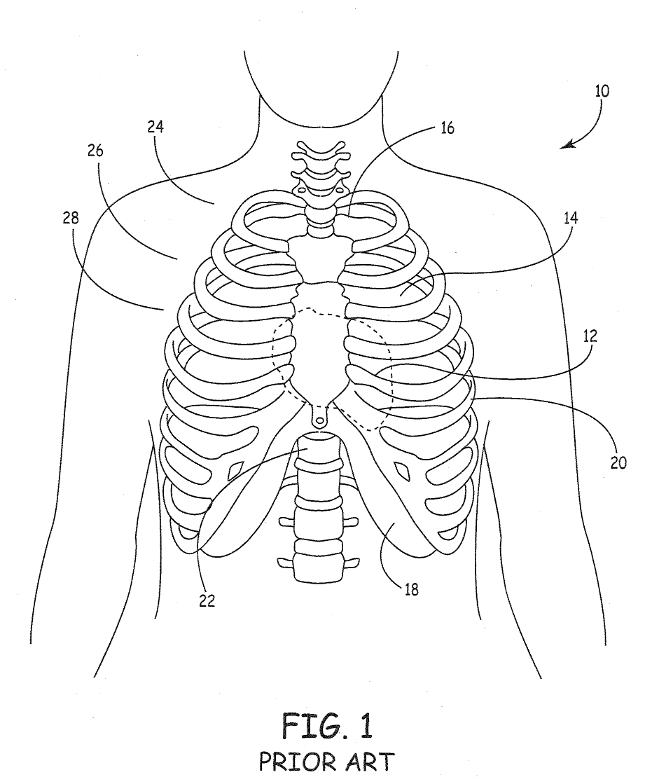

FIG. 1 is a cutaway drawing of patient 10. Heart 12 is positioned in thoracic cavity 14. Thoracic cavity 14 is commonly understood in the art to be bounded by thoracic inlet 16, diaphragm 18, ribs 20 and spine 22. Patient skin 24, musculature 26 and subcutaneous tissue 28 between skin 24 and musculature 26 are commonly not understood to be part of thoracic cavity 14.

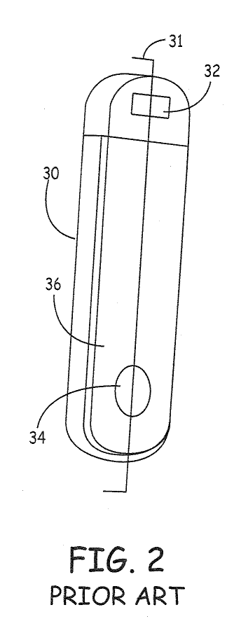

FIG. 2 is an image of implantable device 30. Implantable device 30 may be configured to stratify risk of heart 12 experiencing a cardiac event without meaningful risk of interruption in the collection of patient data and with greater permanence than may be provided with alternative devices, as disclosed, for instance, in U.S. Pat. No. 5,987,352, Klein et al, incorporated herein in its entirety. In various embodiments, implantable device 30 has a length along primary axis 31 from three (3) to six (6) centimeters and has a diameter less than or equal to one (1) inch (2.54 centimeters). In an embodiment, implantable device 30 has a length of approximately four (4) centimeters and a diameter orthogonal to primary axis 31 of one-half (0.5) inch (1.27 centimeters). In various embodiments, implantable device 30 is configured for subcutaneous implantation, which is known in the art to involve implantation of implantable device 30 under skin 24 but outside of thoracic cavity 14 of patient 12. In various embodiments, implantable device 30 may be implanted in tissue 28. Implantable device 30 can also be implanted sub-muscularly, that is below musculature 26, but outside of thoracic cavity 14.

Implantable device 30 may have electrodes 32, 34 at opposing ends of housing 36 along primary axis 31 of implantable device 30. In various alternative embodiments, electrodes 32, 34 are positioned on leads which extend from housing 36. In certain embodiments, the leads are similarly positioned subcutaneously. In alternative embodiments, the leads are transvenous and extend through vasculature of patient 10 and into heart 12. In various embodiments, electrodes 32, 34 are positioned a predetermined distance apart. In an embodiment, the spacing is equal to the length of implantable device 30. In alternative embodiments, electrodes 32, 34 are positioned at a distance of less than the length of implantable device 30. When implanted subcutaneously, electrodes 32, 34 may sense far-field electrical activity of heart 12 which may be interpreted in order to characterize the electrical and physical activity of heart 12.

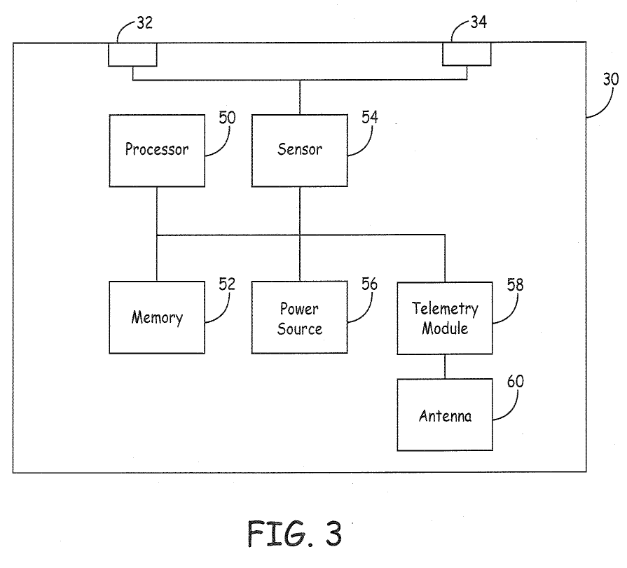

FIG. 3 is a block diagram of implantable device 30. Processor 50 provides computing and controlling functions for implantable device 30. Memory 52 stores data both stored through user input and sensed by implantable device 30 by way of electrodes 32, 34. Sensor 54 is coupled to electrodes 32, 34 and utilizes data sensed by electrodes 32, 34 to identify conditions of heart 12. In various embodiments, the function of sensor 54 is merely an aspect of the overall functionality of processor 50, and as such sensor 54 is not independent circuitry. In alternative embodiments, sensor 54 is separate componentry. Power source 56 provides power to the componentry of implantable device 30. In an embodiment, power source 56 is selected from conventional batteries well known in the implantable medical device art. In alternative embodiments, power source 56 is an alternative source of long-term power, such as a super capacitor. Telemetry module 58 is coupled to antenna 60 which, when placed in proximity of an external receiver, is configured to transmit data from processor 50, memory 52 or sensor 54 to an external device. In an embodiment, antenna 60 is an inductive coil configured to transmit data by way of an inductive field.

As cardiac signals are detected by electrodes 32, 34 and sensed by sensor 54, the data representing the cardiac signals may be stored in memory 52 and/or processed in processor 50. Alternatively, data representing the cardiac signals are transmitted to the external device by way of telemetry module 58 without storage in memory 52 or processing in processor 50. In such embodiments, the external device performs the processing functions.

A measurement of an electrogram detected by electrodes 32, 34 positioned subcutaneously in patient 10 may generally be influenced by a relatively broad region of patient 10. Included in such broad region may be musculature 26 and the lungs of patient 10. Measurements detected with electrodes 32, 34 may be sensitive to signals generated by musculature 26 and lungs, as well as from heart 12, and are commonly referred to as far-field measurements. In various embodiments, electrodes 32, 34 may be positioned in patient 10 so as to replicate or approximately replicate various electrocardiogram vectors known in the art. In an embodiment, one of electrodes 32, 34 may be positioned proximate thoracic inlet 16 and the other of electrodes 32, 34 may be positioned proximate to and slightly below heart 12 in an approximate replication of an electrocardiogram vector known in the art as a V3 vector.

Medical devices such as pacemakers and cardioverter/defibrillators are well known in the art and may incorporate many or all of the componentry of implantable device 30. Broadly speaking, the componentry of implantable device 30 may represent a sensing module or aspect of a pacemaker or cardioverter/defibrillator, with a therapy module incorporated to treat sensed conditions. As such, for the purposes of monitoring the patient, pacemakers and cardioverter/defibrillators known in the art may be adequate to sense, store and analyze cardiac signals in a manner similar to that of implantable device 30.

Similarly, external devices known in the art, such as Holter monitors, may provide sensing and recording capabilities similar to those of implantable device 30. Further, in the case of both external and implantable devices, processing of data may be conducted by external devices which incorporate connectivity to the monitoring device and a processor. Particularly, in the case of the implantable devices, processing external to the patient may conserve implantable resources such as battery life.

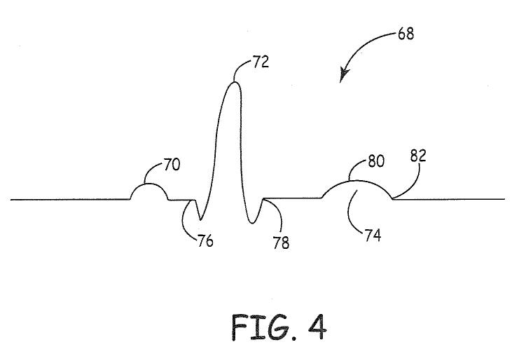

A cardiac complex 68 as detected as part of an electrocardiogram is illustrated in FIG. 4. P-wave 70 represents a depolarization of the atria of heart 12. QRS complex 72 represents a repolarization of the atria of heart 12 and a depolarization of the ventricles of heart 12. T-wave 74 represents the repolarization of the ventricles of heart 12. In the embodiment of implantable device 30, electrodes 32, 34 are configured to detect the electrical signal representative of the cardiac complex and sensing module 54 is configured to interpret the electrical signals sensed by electrodes 32, 34. QRS complex 72 may be defined as the cardiac activity between beginning 76 of the Q-wave and end 78 of the S-wave. T-wave 74 may be identified by way of Tₚₑₐₖ 80 and T_{end} 82.

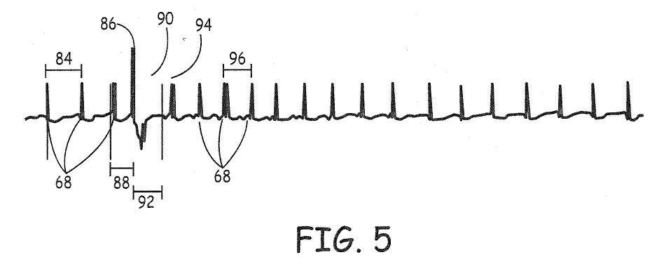

An occurrence premature ventricular contraction, or a PVC as it is known in the art, with an accompanying compensatory pause, is illustrated in exaggerated form in FIG. 5. Normal heartbeats 68 define a baseline heart rate interval 84. Premature ventricular contraction 86 defines interval 88 with the immediately preceding normal heartbeat 68. In various embodiments, premature ventricular contraction 86 is identified when a duration of interval 88 is less than a duration of interval 84 by a predetermined amount. In an embodiment, premature ventricular contraction 86 is identified when the duration of interval 88 is not more than eighty (80) percent of the duration of interval 84.

Compensatory pause 90 may be identified as interval 92 between premature ventricular contraction 86 and compensatory pause beat 94. In various embodiments, compensatory pause 90 may be identified when a duration of interval 92 is greater than the duration of interval 84. In an embodiment, compensatory pause 90 is identified when the duration of interval 92 is at least one hundred twenty (120) percent greater than interval 84. In various alternative embodiments, compensatory pause 90 is identified when the duration of interval 92 is greater by a predetermined percentage than a mathematical function of a predetermined number of intervals 84. In one such embodiment, compensatory pause 90 is identified if interval 92 is at least one hundred twenty (120) percent greater than an average of ten (10) intervals 84.

Following identification of premature ventricular contraction 86 accompanied by compensatory pause 90, the cardiac signal is analyzed for dispersion of repolarizations. In particular, if patient 10 does not have dispersions of repolarization following a premature ventricular contraction 86 accompanied by compensatory pause 90, patient 10 may be at an increased risk of future arrhythmia. In an embodiment, T-wave alternans are analyzed for a predetermined number of cardiac beats 68. As will be described in detail below, the analysis of T-wave alternans may proceed according to several potential methods. Alternatively, as will be described below, dispersions of repolarizations may be determined according to methods unrelated to T-wave alternans.

In an embodiment, dispersion is calculated by comparing T-waves 74 of consecutive cardiac complexes 68 following premature ventricular contraction 86. In particular, Tₚₑₐₖ 80 of the consecutive T-waves 74 is measured and subtracted from one another, with the absolute value of the difference compared against a cutoff threshold. In an alternative embodiment, peak-to-peak amplitude for each T-wave is measured and subtracted. In various embodiments, the cutoff threshold is selected over a range from twenty (20) microvolts to fifty (50) microvolts. In various embodiments, the cutoff threshold is selected from the range of thirty-one (31) microvolts to thirty-seven (37) microvolts. In an embodiment, the cutoff threshold is thirty-four (34) microvolts. If the absolute value of the difference in measured Tₚₑₐₖ values is less than the threshold, accompanied by the identification of premature ventricular contraction 86, compensatory pause 90 and, in an embodiment, an abnormal autonomic reflex, then patient 10 may be identified as not having significant T-wave alternans and, as a result, as being at high risk of future arrhythmia.

In such embodiments, where more than two cardiac complexes 68 are utilized to identify dispersions of repolarizations, consecutive T-waves 74 may be subtracted from one another until the predetermined number of cardiac complexes have been evaluated. In such embodiments, patient 10 may be evaluated as having dispersions of repolarizations if any of the differences between consecutive T-waves 74 are less than the threshold. Alternatively, patient 10 may be evaluated as having dispersions of repolarizations if at least half of the differences are less than the threshold, if the average of the differences is less than the threshold, or if all of the differences are less than the threshold.

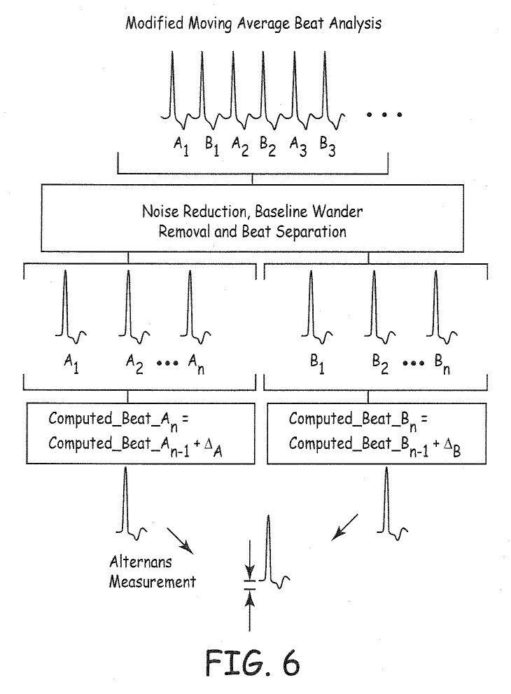

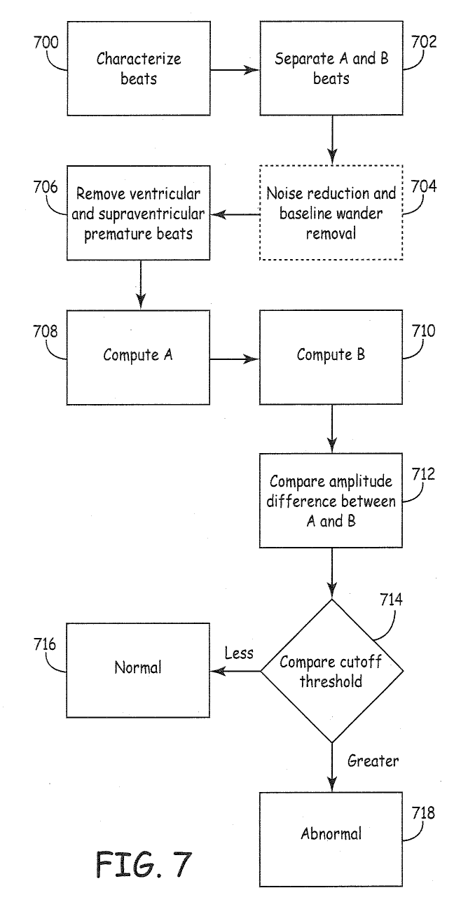

Alternatively, a modified moving average method may be utilized to determine if T-wave alternans suggest dispersions of repolarization. In some embodiments, the T-wave alternans metric utilizes the modified moving average analysis as understood in the art and as described by Nearing, Bruce D. and Verrier, Richard L., in “Modified moving average analysis of T-wave alternans to predict ventricular fibrillation with high accuracy”, J. Appl Physiol 92: 541-549, 2002, which is incorporated herein in its entirety. FIG. 6 illustrates the modified moving average beat analysis method, which is further shown in the flowchart of FIG. 7. Heart beats are alternately characterized (700) as A and B beats. The A and B beats are separated (702) and, in an embodiment, subjected to noise detection, reduction and baseline wander removal (704). Ventricular and supraventricular premature beats are removed (706). All of the A beats are signal averaged to compute (708) a template A beat, and all of the B beats are signal averaged to compute (710) a template B beat. The alternans measurement is obtained by taking (712) the maximum of the absolute difference in amplitude between the computed A template and the computed B template within an ST-T window. In various embodiments, the ST-T window is a window having a duration of two hundred fifty (250) milliseconds starting one hundred fifty (150) milliseconds following beginning 76 of QRS complex 72.

In such embodiments, the cutoff threshold applied above may be compared (714) against the difference. As described above, the cutoff threshold may be selected from the range of fifth (50) microvolts to one hundred fifty (150) microvolts. In various embodiments, the cutoff threshold is selected from the range of ninety (90) microvolts to one hundred (100) microvolts. In an embodiment, the cutoff threshold is 95 microvolts. If the difference is less than the threshold then T-wave alernans are considered normal (716), while if the difference is greater than the threshold the T-wave alternans are considered abnormal (718), with the corresponding implications to the evaluation of the patient's likelihood of future arrhythmias described above with respect to T-wave alternans.

In an embodiment, if a ventricular premature beat is detected during the analysis of T-wave alternans according to FIG. 7, then the pending analysis may be discarded and the process restarted. In various embodiments, detection of a supraventricular beat or premature atrial contraction beat is detected does not result in discarding of the beat or the restart of the process.

In alternative embodiments, the analysis of T-wave alternans to determine dispersions of repolarizations may be replaced with different metrics of the performance of the cardiac substrate. In an embodiment, an integral of a QRST complex, defined as the area under each of QRS complex 72 and T-wave 74, may be computed. In a further embodiment, an area of T-wave 74 may be computed by integrating the T-wave from Tₚₑₐₖ 80 to T_{end} 82. Such a measurement may be indicative of a likelihood that patient 10 will experience fast ventricular tachycardia and/or ventricular fibrillation. A use for T-wave area is described in an abstract by Larisa G. Tereshchenko et al., entitled Tₚₑₐₖ-T_{end} Area Variability Index from Far-Field Implantable Cardioverter-Defibrillator Electrograms Predicts Sustained Ventricular Tachyarrhythmia¹, incorporated here by reference in its entirety. Increased variability of Tₚₑₐₖ-T_{end} area index may provide a measure of both alternating and non-alternating repolarization instability and may indicate dispersions of repolarizations in patient 10. ¹Tereshchenko et. al. “Tpeak-Tend Area Variability Index from Far-Field Implantable Cardioverter-Defibrillator Electrograms Predicts Sustained Ventricular Tachyarrhythmia”, Heart Rhythm, vol 4, no. 5, May Supplement 2007.

Further, a variability in time between QRSₛₜₐᵣₜ 76 to T_{end} 82 may be measured as a Q-T variability index. An example of a use for a Q-T variability index is described in U.S. Pat. No. 5,560,368, Berger, incorporated here by reference in its entirety. A template QT interval may be created based on QRSₛₜₐᵣₜ 76 to T_{end} for one cardiac cycle. An algorithm is then utilized to determine the QT interval of other cardiac cycles by determining how much each cycle must be stretched, i.e. elongated, or compressed in time so as to best match the template.

An abnormal autonomic reflex may be identified on the basis of an occurrence of a predetermined number of premature ventricular contractions 86 over a predetermined time period. In an embodiment, an abnormal autonomic reflex may be identified if not fewer than five (5) premature ventricular contractions 86 occur over a twenty-four (24) hour period. In such an embodiment, the twenty-four (24) hour period is a rolling period, with patient 10 being identified as having an abnormal autonomic reflex providing patient 10 has experienced not fewer than five (5) premature ventricular contractions 86 over the immediately preceding twenty-four (24) hours.

Alternatively, patient 10 may be identified as having an abnormal autonomic reflex based on an identification of heart rate turbulence. Heart rate turbulence refers to the cycle length fluctuations for a number of heart beats following a premature ventricular beat. In various embodiments, the number of beats range from five (5) beats to twenty (20) beats. In an embodiment, the number of beats is sixteen (16) beats. In sinus rhythm, the heart rate may accelerate after the premature beat and then recover to a baseline value over several beats. This adaptation of heart rate to premature ventricular contraction 86 may be absent in high risk patients. Mechanistically, heart rate turbulence may be due to a transient loss of vagal efferent activity due to missed baroreflex afferent input following a premature beat. A drop in blood pressure following premature beat 86 is sensed by a baroreflex receptor of patient 10 which then inhibits a vagal tone of patient 10, resulting in early acceleration of a cardiac cycle length. The inhibition may die out over several beats thereafter and as the blood pressure recovers to normal levels, the baroreflex receptor is reloaded and vagal activity is restored.

Heart rate turbulence is computed from a plot of heart rate intervals 86 and a heart beat number, known in the art as a tachogram. Heart rate turbulence may be characterized by two variables: turbulence onset and turbulence slope. In an embodiment, turbulence onset is defined as the difference between the mean of the first two intervals 96 of consecutive complexes after premature ventricular contraction 86 and the mean of the last two sinus intervals 84 of consecutive complexes preceding premature ventricular contraction 86 divided by the mean of the last two intervals 84 between consecutive complexes preceding premature ventricular contraction 86. In alternative embodiments, turbulence onset may be based on individual intervals 84, 96, or based on more than two intervals 84, 96.

In an embodiment, turbulence slope is defined as the maximum positive slope of a regression line assessed over any sequence of five (5) subsequent sinus-rhythm intervals 96 within the first fifteen (15) sinus-rhythm intervals 96 after premature ventricular contraction 86. In various alternative embodiments, the possible sample set of intervals 96 after a premature ventricular contraction may be as few as two and as many as thirty, while the regression line may be based on a sequence of as few as two (2) subsequent sinus-rhythm intervals 96 and as many intervals 96 as the size of the possible sample set.

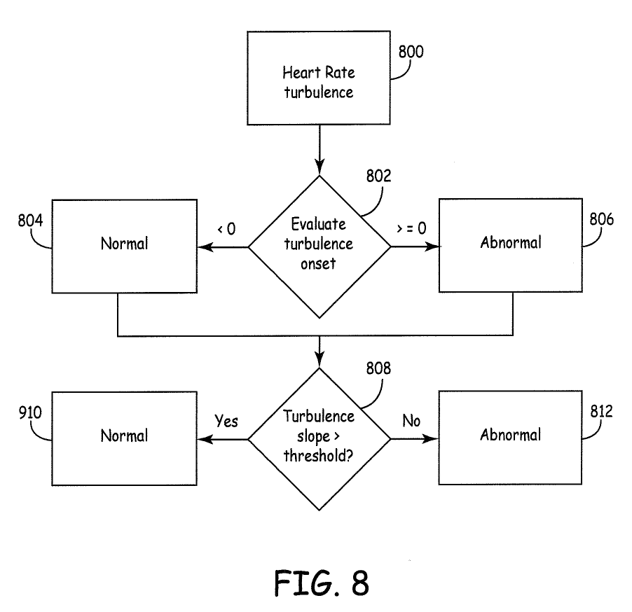

In an embodiment, illustrated in the flowchart of FIG. 8, heart rate turbulence (800) is determined by evaluating (802) a heart rate turbulence onset and evaluates (802) the turbulence onset as normal (804) if the turbulence onset is less than zero, or abnormal (806) if the turbulence onset is greater than or equal to zero, and therefore indicative of increased risk of future arrhythmia. In an embodiment, if the heart rate turbulence is normal (804) the analysis ceases, while if the heart rate turbulence is abnormal (806) then turbulence slope is evaluated. Alternatively, turbulence slope is evaluated in either circumstance.

In an embodiment, if the turbulence slope is greater than or equal to a threshold (808) of 2.5 milliseconds per interval 96, then the turbulence slope is considered normal (810). In alternative embodiments, the threshold may be less than 2.5 milliseconds to provide relatively more stringent requirements for normalcy, and greater than 2.5 milliseconds if the requirements for normalcy may be relatively more relaxed. Otherwise, the turbulence slope is considered abnormal (812). In an embodiment, turbulence slope is the maximum slope of the regression line that fits five (5) intervals 96 during up to thirty (30) beats following a premature ventricular contraction. In alternative embodiments, the regression line may fit more or fewer intervals 96 during more or fewer beats following a premature ventricular contraction. If both heart rate turbulence is evaluated as abnormal (806) and turbulence slope is evaluated as abnormal (812) then patient 10 may be identified as having an abnormal autonomic reflex.

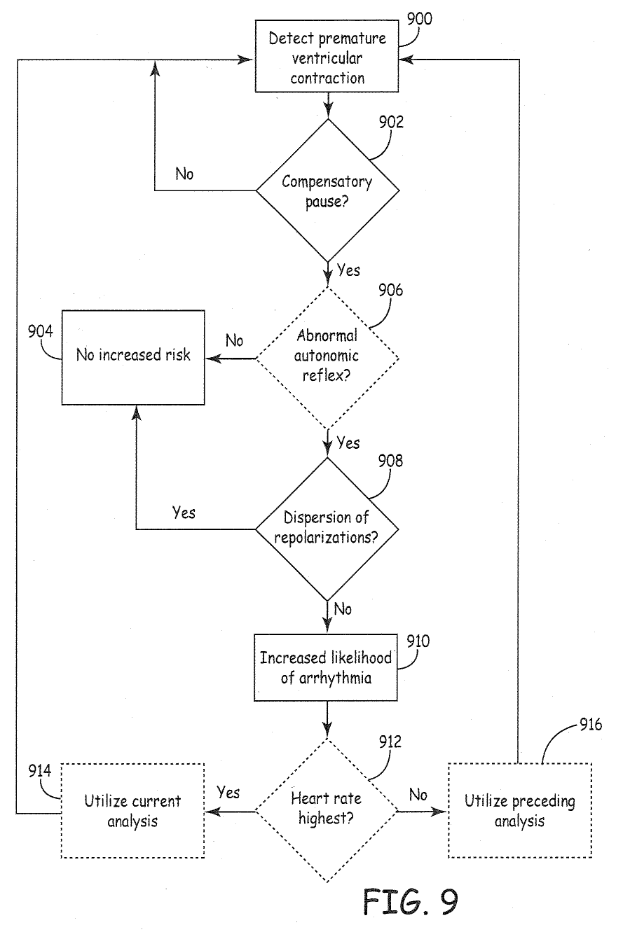

FIG. 9 is a flowchart for identifying patient 10 as being at risk for future arrhythmia. The steps may be performed by processor 50, a processor external to patient 10, or a user. When premature ventricular contraction 86 is detected (900), compensatory pause 90 is checked for (902). If compensatory pause 90 is not detected then an additional premature ventricular contraction 86 may be detected (900). Alternatively, patient 10 may be determined to not be at risk (904) of future arrhythmia. If compensatory pause 90 is detected, in an optional embodiment, it is determined (906) if patient 10 has an abnormal autonomic reflex. If patient 10 does not have an abnormal autonomic reflex patient 10 may be determined not to be at high risk (904) of future arrhythmia. If patient 10 does have an abnormal autonomic reflex, it is determined (908) if patient 10 has dispersions of repolarizations. In an alternative optional embodiment, even if patient 10 does not have an abnormal autonomic reflex it is determined (908) if patient 10 has dispersions of repolarizations.

If patient 10 does not have dispersions of repolarization then patient 10 may be evaluated as having an increased likelihood of suffering a future arrhythmia (910), while if patient 10 does have dispersions of repolarization patient 10 may be evaluated as not having a likelihood of future arrhythmias (904). In further alternative embodiments, it is determined (906) if patient 10 has an abnormal autonomic reflex and it is determined (908) if patient has dispersions of repolarizations concurrently. In such embodiments, if patient 10 has either an abnormal autonomic reflex or lack of dispersions of repolarizations, patient may be evaluated as having a likelihood of future arrhythmias (910), while if patient 10 has neither an abnormal autonomic reflex nor lack of dispersions of repolarizations then patient 10 may not have a likelihood of future arrhythmias (904).

In various embodiments consistent with the flowchart of FIG. 9, determining whether patient 10 has an abnormal autonomic reflex (906) may occur at varying places in the flowchart. Such places include before detecting (900) premature ventricular contraction 86 and concurrent with determining whether patient 10 has dispersions of repolarizations (908). In particular, determination of abnormal autonomic reflex (906) may be conducted before detecting (900) premature ventricular contraction 86. In addition, risk of future arrhythmias may be based on a frequency of premature ventricular contractions 86 over time, i.e., the number of premature ventricular contractions 86 detected over the predetermined period of time exceeds the predetermined required number. Alternatively, if the metric for heart rate turbulence is utilized to identify an abnormal autonomic reflex, the cardiac complexes 68 used to identify heart rate turbulence may be coincident, at least in part, with the cardiac complexes utilized to identify dispersions of repolarizations.

In various alternative embodiments, analysis of likelihood of future arrhythmia is conducted iteratively. During the cardiac complexes 68 utilized to identify the dispersions of repolarizations the heart rate of patient 10 may be measured and recorded along with the results of the analysis (908). In such embodiments, the measured heart rate may be compared against other heart rates measured during preceding analyses originating from previous premature ventricular contraction determinations (900). It has been determined that the dispersions of repolarizations analysis corresponding to the highest heart rate during each individual analysis (908) may be relatively more indicative of patient risk than dispersions of repolarizations analyses (908) conducted with relatively lower heart rates. As such, for each new analysis (908), the measured heart rate may be compared (912) against the highest preceding measured heart rate from previous analyses (908). If the current measured heart rate is greater than the preceding highest measured heart rate, then the current analysis is stored and utilized (914) to identify patient's 10 likelihood of future arrhythmia. If the current measured heart rate is less than the preceding highest measured heart rate then the current analysis is discarded and the analysis corresponding to the highest rate is utilized (916) to identify patient's 10 likelihood of future arrhythmia. Upon completing the analysis the system returns to iteratively detecting premature ventricular contraction 86 (900).

In an embodiment, if a subsequent premature ventricular contraction occurs during the subsequent beats following premature ventricular contraction 86 and ventricular pause 90, then the method of FIG. 9 is aborted, the subsequent premature ventricular contraction is selected for analysis and the flowchart restarted.

In various embodiments, the method illustrated in the flowchart of FIG. 9 may be implemented on any patient 10 at any time and produce indications of a likelihood of future arrhythmia. Certain embodiments, such as those including analysis of patient's 10 autonomic reflex, may be particularly useful in analyzing a condition of a patient 10 who has recently suffered a myocardial infarction. Such an analysis may be indicative of patient's 10 likelihood of recovering from the myocardial infarction without suffering from an arrhythmia.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A system for assessing a likelihood of a patient having a heart to experience a cardiac arrhythmia, comprising:
a sensor configured to detect a premature ventricular contraction of said heart, said premature ventricular contraction being premature relative to a rate of ventricular contractions during a period preceding said premature ventricular contraction; and
a processor, operatively coupled to said sensor, configured to:
determine a dispersion of a predetermined number of repolarizations of said heart of said patient occurring following said premature ventricular contraction of said heart if a compensatory pause is detected in conjunction with said premature ventricular contraction; then
determine said likelihood of said patient experiencing said cardiac arrhythmia based, at least in part, on said dispersion of said plurality of repolarizations of said heart of said patient.

2. The system of claim 1 wherein said likelihood of said patient experiencing said cardiac arrhythmia is relatively large when said dispersion of said predetermined number of repolarizations is relatively small.

3. The system of claim 1 wherein said predetermined number of repolarizations occur immediately following said premature ventricular contraction of said heart.

4. The system of claim 1 wherein said predetermined number of repolarizations are a predetermined number of T-waves of a cardiac complex of said heart, and wherein said determining a dispersion step is based, at least in part, on an alternating characteristic of said predetermined number of T-waves.

5. The system of claim 1 wherein said predetermined number of premature ventricular contractions is at least five over a period of time of approximately twenty-four hours.

6. The system of claim 1 wherein said processor is further configured to return to detecting said premature ventricular contraction after determining said likelihood to detect further premature ventricular contractions;
wherein said processor determines said dispersion of said predetermined number of repolarizations of said heart following each of said premature ventricular contractions of said heart;
wherein said processor is further configured to determine said likelihood further based, at least in part, on said dispersion of said predetermined number of said plurality of repolarizations of said heart of said patient when said heart rate is relatively higher to the exclusion of said dispersion of said predetermined number of said plurality of repolarizations of said heart of said patient occurring when said heart is relatively lower.

7. The system of claim 1 wherein said processor is configured to return to detecting said premature ventricular contraction after determining said likelihood to detect further premature ventricular contractions;
wherein said processor determines said dispersion of said predetermined number of repolarizations of said heart following each of said premature ventricular contractions of said heart;
wherein said processor is further configured to determine said likelihood further based, at least in part, on an average of said dispersion of said predetermined number of said plurality of repolarizations of said heart of said patient when said heart rate taken over a plurality of a predetermined number of repolarizations of said heart of said patient.

8. The system of claim 1 wherein said processor detects said premature ventricular contraction based, at least in part, on an interval between an R-wave of said premature ventricular contraction and an R-wave of an immediately preceding cardiac beat being not more than approximately eighty percent of an interval corresponding to said rate of ventricular contractions during a period preceding said premature ventricular contraction.

9. The system of claim 8 wherein said rate of ventricular contractions is based, at least in part, on an interval between R-waves of two cardiac beats immediately preceding said premature ventricular contraction.

10. The system of claim 8 wherein said rate of ventricular contractions is based, at least in part, on an average interval between R-waves preceding said premature ventricular contraction.

11. The system of claim 1 wherein said premature ventricular contraction is associated with said compensatory pause if an interval between an R-wave of said premature ventricular contraction and an R-wave of a cardiac beat immediately following said premature ventricular contraction is not less than approximately one hundred twenty percent of an interval corresponding to said rate of ventricular contractions during a period preceding said premature ventricular contraction.

12. The system of claim 11 wherein said rate of ventricular contractions is based, at least in part, on an interval between R-waves of two cardiac beats immediately preceding said premature ventricular contraction.

13. The system of claim 11 wherein said rate of ventricular contractions is based, at least in part, on an average interval between R-waves preceding said premature ventricular contraction.

14. The system of claim 1 wherein said predetermined number of depolarizations is within four of sixteen.

15. The system of claim 1 wherein said predetermined number of depolarizations is sixteen.

16. The system of claim 1 wherein said processor is configured to determine an autonomic tone of said heart and perform said determining only if said autonomic tone is present.

17. The system of claim 16 wherein said autonomic tone is based, at least in part, on an elevated heart rate said patient relative to a baseline heart rate.

18. The system of claim 17 wherein said elevated heart rate is measured during said predetermined number of repolarizations of said heart of said patient.

19. The system of claim 18 wherein said elevated heart rate is determined by an average of heart rate during said predetermined number of repolarizations of said heart of said patient.

20. The system of claim 18 wherein said elevated heart rate is determined by a highest heart rate occurring during said predetermined number of repolarizations of said heart of said patient.

21. A system for assessing a likelihood of a patient having a heart to experience a cardiac arrhythmia, comprising:
a sensor configured to sense T-wave changes following a premature ventricular contraction of said heart that introduces a baroreflex of said heart, said premature ventricular contraction being premature relative to a rate of ventricular contractions during a period preceding said premature ventricular contraction; and
a processor configured to determine said likelihood of said patient experiencing said cardiac arrhythmia with said processor based, at least in part, on an alternating characteristic of a predetermined number of T-waves following said premature ventricular contraction.

22. A method for assessing a likelihood of a patient to experience a cardiac arrhythmia with a system comprising a sensor and a processor, comprising the steps of:
detecting a premature ventricular contraction of said heart, said premature ventricular contraction being premature relative to a rate of ventricular contractions during a period preceding said premature ventricular contraction; then
if a compensatory pause is detected in conjunction with said premature ventricular contraction, determining a dispersion of a predetermined number of repolarizations of said heart of said patient occurring following said premature ventricular contraction of said heart; then
determining said likelihood of said patient experiencing said cardiac arrhythmia with said processor based, at least in part, on said dispersion of said plurality of repolarizations of said heart of said patient.

23. The method of claim 22 wherein said likelihood of said patient experiencing said cardiac arrhythmia is relatively large when said dispersion of said predetermined number of repolarizations is relatively small.

24. The method of claim 22 wherein said predetermined number of repolarizations occurs immediately following said premature ventricular contraction of said heart.

25. The method of claim 22 wherein said predetermined number of repolarizations are said predetermined number of T-waves of a cardiac complex of said heart, and wherein said determining a dispersion step is based, at least in part, on an alternating characteristic of said predetermined number of T-waves.

26. The method of claim 22 wherein said predetermined number of premature ventricular contractions is at least five over a period of time of approximately twenty-four hours.

27. The method of claim 22, further comprising the step of:
after said determining said likelihood step, returning to said detecting said premature ventricular contraction step to detect further premature ventricular contractions;
wherein said dispersion of said predetermined number of repolarizations of said heart is determined following each of said premature ventricular contractions of said heart;
wherein said determining said likelihood step is further based, at least in part, on said dispersion of said predetermined number of said plurality of repolarizations of said heart of said patient when said heart rate is relatively higher to the exclusion of said dispersion of said predetermined number of said plurality of repolarizations of said heart of said patient occurring when said heart is relatively lower.

28. The method of claim 22, further comprising the step of:
after said determining said likelihood step, returning to said detecting said premature ventricular contraction step to detect further ventricular contractions;
wherein said dispersion of said predetermined number of repolarizations of said heart is determined following each of said premature ventricular contractions of said heart;
wherein said determining said likelihood step is further based, at least in part, on an average of said dispersion of said predetermined number of said plurality of repolarizations of said heart of said patient when said heart rate taken over a plurality of a predetermined number of repolarizations of said heart of said patient.

29. The method of claim 22 wherein said detecting said premature ventricular contraction step is based, at least in part, on an interval between an R-wave of said premature ventricular contraction and an R-wave of an immediately preceding cardiac beat being not more than approximately eighty percent of an interval corresponding to said rate of ventricular contractions during a period preceding said premature ventricular contraction.

30. The method of claim 29 wherein said rate of ventricular contractions is based, at least in part, on an interval between R-waves of two cardiac beats immediately preceding said premature ventricular contraction.

31. The method of claim 29 wherein said rate of ventricular contractions is based, at least in part, on an average interval between R-waves preceding said premature ventricular contraction.

32. The method of claim 22 wherein said premature ventricular contraction is associated with said compensatory pause if an interval between an R-wave of said premature ventricular contraction and an R-wave of a cardiac beat immediately following said premature ventricular contraction is not less than approximately one hundred twenty percent of an interval corresponding to said rate of ventricular contractions during a period preceding said premature ventricular contraction.

33. The method of claim 32 wherein said rate of ventricular contractions is based, at least in part, on an interval between R-waves of two cardiac beats immediately preceding said premature ventricular contraction.

34. The method of claim 32 wherein said rate of ventricular contractions is based, at least in part, on an average interval between R-waves preceding said premature ventricular contraction.

35. The method of claim 22 wherein said predetermined number of depolarizations is within four of sixteen.

36. The method of claim 22 wherein said predetermined number of depolarizations is sixteen.

37. The method of claim 22 further comprising the step of determining an autonomic tone of said heart and performing said determining step occurs only if said autonomic tone is present.

38. The method of claim 37 wherein said autonomic tone is based, at least in part, on an elevated heart rate of said patient relative to a baseline heart rate.

39. The method of claim 38 wherein said elevated heart rate is measured during said predetermined number of repolarizations of said heart of said patient.

40. The method of claim 39 wherein said elevated heart rate is determined by an average of heart rate during said predetermined number of repolarizations of said heart of said patient.

41. The method of claim 39 wherein said elevated heart rate is determined by a highest heart rate occurring during said predetermined number of repolarizations of said heart of said patient.

42. A method for assessing a likelihood of a patient having a heart to experience a cardiac arrhythmia with a system comprising a sensor and a processor, comprising the steps of:
evaluating T-wave changes following a premature ventricular contraction of said heart that introduces a baroreflex of said heart, said premature ventricular contraction being premature relative to a rate of ventricular contractions during a period preceding said premature ventricular contraction; and then
determining said likelihood of said patient experiencing said cardiac arrhythmia with said processor based, at least in part, on an alternating characteristic of a predetermined number of T-waves following said premature ventricular contraction.
